(19) United States
(12) Patent Application Publication
(10) Pub. No.: US 2006/0205974 A1
(43) Pub. Date: Sep. 14, 2006

(54) Processes for producing aromatic dicarboxylic acids

(76) Inventors: Gino Georges Lavoie, Kingsport, TN (US); Robert Thomas Hembre, Johnson City, TN (US); Charles Edwan Sumner JR., Kingsport, TN (US); Joseph Nathaniel Bays, Kingsport, TN (US); Daniel Burts Compton, Kingsport, TN (US); Brent Alan Tennant, Kingsport, TN (US); Bryan Wayne Davenport, Columbia, SC (US); David Lange, Irmo, SC (US); Thomas Richard Floyd, Kingsport, TN (US); Stephan Andreas Schunk, Heidelberg (DE); Oliver Busch, Recklinghausen (DE)

Correspondence Address: Michael K. Carrier;Eastman Chemical Company, P.O. Box 511, Kingsport, TN, 37662-5075, US

(21) Appl. No.: 11/075,008

(22) Filed: Mar. 8, 2005

(51) Int. Cl. C07C 51/16; C07C 51/255

(52) U.S. Cl. 562/410

(57) ABSTRACT

Processes for the liquid phase oxidation of one or more of a para- or a meta-substituted dialkyl aromatic compound are disclosed, the processes including a step of combining in a reaction medium the one or more para- or meta-substituted dialkyl aromatic compound, a solvent mixture comprising water and a saturated organic acid having from 2-4 carbon atoms, and an oxygen-containing gas, at a temperature from about 130° C. to about 180° C., in the presence of a catalyst composition comprising cobalt, manganese, bismuth, and bromine. The processes produce the corresponding aromatic dicarboxylic acid product with improved conversion, while reducing the formation of carbon oxides.

### FIELD OF THE INVENTION

This invention pertains to improved processes for the production of para- and meta-substituted aromatic dicarboxylic acids by the liquid-phase oxidation of the corresponding para- and meta-substituted dialkyl aromatic hydrocarbons, the processes resulting in improved conversion with reduced formation of carbon oxides.

### BACKGROUND OF THE INVENTION

Aromatic dicarboxylic acids, and especially terephthalic acid and isophthalic acid, are used to produce a variety of polyester products, important examples of which are poly(ethylene terephthalate) and its copolymers. These aromatic dicarboxylic acids may be synthesized by the catalytic oxidation of the corresponding dialkyl aromatic compound. For example, terephthalic acid (TPA) and isophthalic acid (IPA) may be produced by the liquid phase oxidation of p-xylene and m-xylene, respectively.

These processes typically comprise feeding one or more dialkyl aromatic hydrocarbons, fresh and/or recycled solvent or reaction medium, and catalyst components to a reactor to which a molecular oxygen-containing gas also is fed, typically near the bottom of the reactor. Conventional liquid-phase oxidation reactors are equipped with agitation means for mixing the multi-phase reaction medium. Agitation of the reaction medium is supplied in an effort to promote dissolution of molecular oxygen into the liquid phase of the reaction medium, and to facilitate contact between the dissolved oxygen and the dialkyl aromatic hydrocarbon in the reaction medium. Agitation of the reaction medium undergoing liquid-phase oxidation is frequently provided by mechanical agitation means in vessels such as, for example, continuous stirred tank reactors (CSTRs). Bubble column reactors provide an attractive alternative to CSTRs and other mechanically agitated oxidation reactors.

In these processes, bubble column reactors may be used having relatively high height to diameter ratios. The oxygen-containing process gas rising through the liquid contents of the reactor results in agitation of the reaction mixture. Alternatively, continuous stirred tank reactors may be used, typically having a lower height to diameter ratio than bubble column reactors. The aromatic dicarboxylic acid produced may be removed continuously through an exit port as a slurry. Process gas containing excess oxygen, along with solvent decomposition products, may be removed through an upper exit port typically located at or near the top of the reactor. The heat of reaction may also be removed through the upper exit port by vaporization of the process solvent and water generated by the reaction.

Thus, in one example of such a process, p-xylene is oxidized to produce terephthalic acid. The p-xylene may be continuously or batchwise oxidized in the primary oxidation reactor in the liquid phase, in the presence of an oxygen-containing gas such as air. In such a process, p-xylene, an oxidation catalyst composition, a molecular source of oxygen, and a solvent such as aqueous acetic acid are combined as a reaction medium in the reactor to produce a crude terephthalic acid (CTA) reaction product. Typical oxidation catalyst compositions include a cobalt compound and a manganese compound, usually in combination with a promoter such as a bromine compound. See, for example, U.S. Pat. Nos. 2,833,816, 3,089,906, and 4,314,073, the disclosures of which are incorporated herein by reference. The process conditions are highly corrosive due to the acetic acid and bromine, and titanium is typically used in the process equipment. See, for example, U.S. Pat. No. 3,012,038, incorporated herein by reference. Acetaldehyde may be used as a promoter in place of bromine, in which case titanium materials need not be used. Acetaldehyde is also useful as an initiator. Because the liquid-phase oxidations of dialkyl aromatic compounds just described are highly exothermic reactions, they are commonly carried out in vented reaction vessels, the heat of reaction being removed by vaporization of the process solvent through the upper exit port.

The resulting CTA is not very soluble in the acetic acid solvent under the reaction conditions, and precipitates from the solvent to form a suspension. This crude terephthalic acid suspension includes terephthalic acid solids, a solvent acting as the suspending medium for the solids and containing a small amount of dissolved terephthalic acid; catalyst components; unreacted p-xylene; incompletely oxidized intermediate oxidation products such as para-tolualdehyde (p-TAl), para-toluic acid (p-TA), and 4-carboxybenzaldehyde (4-CBA); and organic impurities such as fluorenones that are known to cause discoloration. The crude terephthalic acid composition is discharged from the oxidation zone and subjected to any of several mother liquor exchange, separation, purification, or recovery methods, with the recovered solvent and catalyst composition being recycled directly back to the oxidation reaction or after processing, such as by catalyst recovery or solvent purification. It is desirable to minimize the amount of incompletely oxidized intermediates and the colored impurities, to reduce the subsequent purification requirements.

Other by-products of the liquid phase oxidation which are partially or completely removed from the reaction mixture in the oxidation reactor are the off-gases, which include water, solvent, unreacted oxygen and other unreacted gases found in the source of the molecular oxygen gas such as nitrogen and carbon dioxide, and additional amounts of carbon dioxide and carbon monoxide that are oxidative losses resulting in part from the catalytic decomposition of the solvent and other oxidizable compounds under the oxidation conditions. The off-gases are vented at the overhead of the oxidation reactor to a distillation column or a condenser to separate the solvent from the other off-gases such as water, carbon dioxide, carbon monoxide, nitrogen, gaseous bromine compounds such as methyl bromide, etc.

Although it is desirable to recover and recycle as much solvent as possible, the solvent is oxidatively decomposed to some extent into its constituent gaseous products, carbon dioxide and carbon monoxide, requiring a fresh source of make-up solvent. This oxidative decomposition is often referred to in the industry as solvent burn or acid burn, and is generally believed to be responsible in part for the formation of carbon oxides, although a portion of the carbon oxides produced is also the result of oxidative decomposition of the dialkyl aromatics or intermediate reaction products. Controlling or reducing formation of carbon oxides would significantly lower the operating costs of the oxidation process, by allowing a greater amount of solvent to be recovered and recycled back to the oxidation zone, and by reducing yield loss from the oxidative decomposition of the aromatic reactants. However, a reduction in carbon oxides formation should not come at the expense of significantly reduced yield or conversion, or an increase in the amount of incomplete oxidation products in the crude mixture, and if possible, it would be desirable to simultaneously reduce carbon oxides formation and increase the conversion. Typically, however, increased conversion is accompanied by an increase in carbon oxides formation.

U.S. Pat. No. 3,931,304 discloses that the addition of non-transitional bismuth ion to oxidation catalysis provided by heavy, transition metal-bromine ion combinations containing both cobalt and manganese ions increases the catalytic activity of the combination for converting the ortho-substituted methyl groups to the corresponding carboxylic acid groups on a benzene nucleus by a factor much greater than by the addition of equivalent amounts of the cobalt and manganese. The greater catalytic activity is said to be manifested by a longer sustained initial rapid rate of oxygen consumption and higher o-dicarboxybenzene yield. This effect was not observed in the oxidation of xylenes having no o-methyl groups, such as m-xylene and p-xylene.

There remains a need in the art for aromatic oxidation processes for the conversion of para- or meta-substituted dialkyl aromatic compounds to the corresponding dicarboxylic acids that achieve improved conversion, while minimizing carbon oxides formation. These and additional advantages are obtained by the present invention, as further described below.

### SUMMARY OF THE INVENTION

The invention relates to processes for the liquid phase oxidation of para- and meta-substituted dialkyl aromatics, to produce the corresponding para- or meta-substituted aromatic dicarboxylic acids, the processes comprising combining in a reaction medium the para- and/or meta-substituted dialkyl aromatic, for example p-xylene or m-xylene, an aqueous solvent comprising one or more saturated organic acids having from 2-4 carbon atoms, and an oxygen-containing gas, at a temperature from about 130° C. to about 180° C., in the presence of a catalyst composition comprising cobalt atoms, manganese atoms, bismuth atoms, and bromine atoms.

According to the invention, when bismuth is added to a catalyst composition comprising cobalt, manganese, and bromine, improved conversion is obtained at moderate temperatures without an unacceptable increase in carbon oxides formation.

### BRIEF DESCRIPTION OF THE DRAWINGS

FIG. 1 illustrates a process flow of crude terephthalic acid streams and the overhead of an oxidation unit.

### DETAILED DESCRIPTION OF THE INVENTION

The present invention may be understood more readily by reference to the following detailed description of the invention, including the appended figure, and to the examples provided. It is to be understood that the terminology used is for the purpose of describing particular embodiments only and is not intended to be limiting.

As used in the specification and the claims, the singular forms “a,” “an,” and “the” include plural referents unless the context clearly dictates otherwise. Thus, when the disclosure and the claims use the phrase “a dialkyl aromatic,” the phrase is intended to encompass one or more dialkyl aromatics. Similarly, when the phrase “an organic acid having from 2-4 carbon atoms” is used, for example, the phrase is intended to encompass one or more such organic acids.

It is to be understood that the words “comprising” and “containing” are open ended and may include any number and type of unstated steps, processes, or ingredients. The description of method steps does not preclude intervening steps and is not restricted to carrying out the steps in a particular order unless otherwise stated. Numerical ranges include each integer and all fractions thereof between the end points of the stated range.

Unless otherwise indicated, the weight amount of catalyst is based in each instance on the total weight of the liquid in the reaction medium, without regard to the amount of precipitated product in the reaction medium, the amount of which may change during the course of the reaction, especially in those cases in which the process is carried out as a batch or semi-batch process. The defined weight amounts may be determined by removal of a portion of the reaction medium either during or after the reaction, since the amount present in the reaction mixture may differ somewhat from the concentration of catalyst as initially provided to the reaction mixture, due to evaporation, solvent burn, etc.

The invention relates to processes for the liquid phase oxidation of one or more of a para- or a meta-substituted dialkyl aromatic compound, which for discussion purposes may be described herein simply as dialkyl aromatics, it being understood that the para- and/or the meta-substituted aromatics are intended to be described. The invention thus includes oxidizing either a para- or a meta-substituted dialkyl aromatic in the alternative, oxidizing both a para- and a meta-substituted dialkyl aromatic together, or even mixtures of more than one para-substituted, or more than one meta-substituted, dialkyl aromatic. Finally, mixtures of more than one para-substituted dialkyl aromatic, in combination with more than one meta-substituted dialkyl aromatic, may also be oxidized together in the liquid phase according to the present invention.

In the processes according to the invention, the extent of carbon oxides formation observed is minimized, for example, in the oxidation of p-xylene to terephthalic acid or m-xylene to isophthalic acid, when bismuth is added to a catalyst system comprising cobalt, manganese, and bromine. The use of the catalyst compositions according to the invention thus leads to low quantities of CO and CO₂ produced in the course of the reaction, which is believed to be a good indicator of the extent of acid burn. Such a decrease in carbon oxides formation in the oxidation of p-xylene or m-xylene translates into significant cost savings in the manufacture of terephthalic acid or isophthalic acid, as the case may be. Remarkably, the addition of bismuth improves conversion while having very little impact on carbon oxides formation.

Thus, according to the invention, one or more para- or meta-substituted dialkyl aromatic compounds, provided as a liquid, preferably p-xylene and/or m-xylene, is oxidized in an aqueous aliphatic solvent, such as acetic acid and water, with oxygen-containing gas, in the presence of a catalyst system comprising cobalt atoms, manganese atoms, bismuth atoms, and bromine atoms. The processes may be carried out at temperatures, for example, from about 130° C. to about 180° C., or from about 135° C. to about 175° C., or from about 140° C. to about 165° C.

According to the invention, the bismuth atoms may be present in an amount, for example, of at least about 25 ppm, or at least 50 ppm, or at least 100 ppm, up to about 1,100 ppm, or up to about 1,500 ppm, or up to about 1,800 ppm.

The processes according to the invention produce one or more aromatic dicarboxylic acids as a reaction product, with good conversions. For example, in those embodiments in which p-xylene is the reactant, a concentration of 4-carboxybenzaldehyde (4CBA) is achieved, based on the weight of aromatic dicarboxylic acids produced, which is typically less than about 8 wt. %, or less than about 6 wt. %, or less than about 3 wt. %, or even 1 wt. % or less. Similarly, when m-xylene is the reactant, a concentration of 3-carboxybenzaldehyde (3CBA) is achieved, based on the weight of aromatic dicarboxylic acids produced, which is typically less than about 8 wt. %, or less than about 6 wt. %, or less than about 3 wt. %, or even 1 wt. % or less. When the reactant comprises a mixture of the para- and meta-substituted dialkyl aromatics, the total amount of the corresponding carboxybenzaldehydes formed will typically be less than about 8 wt. %, or less than about 6 wt. %, or less than about 3 wt. %, or even 1 wt. % or less.

The amount of carbon oxides formation in the processes according to the invention will typically be no more than about 1.2 moles COₓ, or no more than about 0.6 mole COₓ, or no more than about 0.3 mole COₓ, in each case with respect to the molar quantity of dialkyl aromatic compounds fed to the reactor. COx values are defined as the total values of carbon monoxide and carbon dioxide.

Thus, in one embodiment, the process comprises oxidizing a para- or meta-substituted dialkyl aromatic in the liquid phase. The liquid phase may at any moment comprise any or all of: the para- or meta-substituted dialkyl aromatic, the oxygen-containing gas, the solvent, the catalyst composition, and the corresponding para- or meta-substituted dicarboxylic acid reaction product dissolved or suspended in the reaction mixture, especially when the process is carried out as a continuous process. The products of the processes according to this embodiment include the dicarboxylic acid solids as the predominant product (for example, at least 50 wt. % of the solids), and incomplete oxidation products which may be found in the solids, in the liquid phase, or in both. The dialkyl aromatic fed to the oxidation reactor may be purified of contaminants which may interfere with the oxidation reaction. The reactant feed may be pure or a mix of the compound isomers or lower or higher homologues, as well as some saturated alicyclic or aliphatic compounds having similar boiling points to the aromatic or fused ring compounds. However, in this embodiment, at least 80 wt. %, preferably at least 95 wt. %, or at least 98 wt. % of the liquid reactants is the para- or meta-substituted dialkyl aromatic reactant.

A number of para- or meta-substituted dialkyl aromatic reactants may be used according to the invention. p-xylene or m-xylene, or both, are particularly useful reactants according to the invention, and may together or separately comprise at least 50 wt % of the dialkyl aromatic compounds provided to the reaction medium, or at least 60 wt. %, or at least 80 wt. %, or at least 90 wt. %, or even at least 95 wt. % or 99 wt. % or more, of the total amount of dialkyl aromatic reactants provided to the reaction medium. para- or meta-substituted naphthalene compounds are also suitable for use according to the invention, as further described below.

In various embodiments, the para- and/or meta-substituted dialkyl aromatic compounds provided to the reaction medium may comprise at least 50 wt. % of the dialkyl aromatic compounds provided to the reaction medium, or at least 60 wt. %, or at least 75 wt. %, or at least 90 wt. %, or at least 95 wt. % or more, in each case based on the total weight of the dialkyl aromatic compounds provided to the reaction medium. In those embodiments in which the para- and meta-substituted dialkyl aromatic compounds do not together comprise substantially all of the dialkyl aromatic compounds provided to the reaction medium, the reaction medium may include ortho-substituted dialkyl aromatic compounds, although such compounds may not in all instances be desired or preferred. Thus, in a preferred embodiment, the para- and/or the meta-substituted dialkyl aromatic compounds comprise substantially all of the dialkyl aromatic compounds provided to the reaction medium.

According to the invention, the liquid phase oxidation processes are carried out in the presence of an aliphatic solvent. Suitable solvents are those which are solvents for the dialkyl aromatics under the oxidation reaction conditions, and especially those in which the dicarboxylic acid products form a pumpable crude flow discharged from the oxidation reactor. Suitable solvents include mixtures of water and the aliphatic solvents. The preferred aliphatic solvents are aliphatic carboxylic acids, and include aqueous solutions of C₂ to C₆ monocarboxylic acids, and preferably C₂ to C₄ monocarboxylic acids, e.g., acetic acid, propionic acid, n-butyric acid, isobutyric acid, n-valeric acid, trimethylacetic acid, caprioic acid, and mixtures thereof. Preferably, the solvent is volatile under the oxidation reaction conditions to allow it to be taken as an off-gas from the oxidation reactor. It is also preferred that the solvent selected is one in which the catalyst composition is soluble under the reaction conditions.

A preferred solvent for use according to one embodiment of the invention, in which the dialkyl aromatic compound comprises para-xylene, is an aqueous acetic acid solution, having a concentration, for example, from about 90 to about 97 wt. % acetic acid, based on the weight of the liquid phase of the reaction medium. In various embodiments, the solvent comprises a mixture of water and acetic acid which, for example, has a water content sufficient to provide at least about 3% by weight water in the reaction medium, or at least 4 wt. %, or from about 3 wt. % to about 15 wt. %, or from 3 wt. % to 11 wt. %.

A preferred solvent for use according to another embodiment of the invention, in which the dialkyl aromatic compound comprises meta-xylene, is an aqueous acetic acid solution, having a concentration, for example, from about 85 to about 97 wt. % acetic acid, based on the weight of the liquid phase of the reaction medium. In various embodiments, the solvent comprises a mixture of water and acetic acid which, for example, has a water content sufficient to provide at least about 3% by weight water in the reaction medium, or at least 6 wt. %, or from about 8 wt. % to about 15 wt. %, or from 3 wt. % to 13 wt. %.

The crude dicarboxylic acid composition may be discharged from the oxidation zone and subjected to a variety of mother liquor exchange, separation, purification, or recovery methods. These methods can provide recovered solvent and catalyst composition for recycling back to the oxidation zone.

Thus, a portion of the solvent feed to the primary oxidation reactor may be obtained from a recycle stream obtained by displacing, for example, from about 80 to 90% of the mother liquor taken from the crude reaction mixture stream discharged from the primary oxidation reactor with fresh, wet acetic acid. This exchange may be accomplished in any convenient apparatus but can most easily be accomplished in a centrifuging apparatus, such as one or more cyclones.

The processes according to the invention are conducted in the presence of a source of oxygen. This may be accomplished by feeding an oxygen-containing gas to the oxidation reactor to allow the gas to contact the liquid reaction mixture in the reactor. The predominately gas-phase oxidant stream introduced into the reactor comprises molecular oxygen (O₂), for example in the range from about 5 to about 100 mole percent molecular oxygen, or from about 10 to about 50 mole percent molecular oxygen, or from 15 to 25 mole percent molecular oxygen. The balance of the oxidant stream typically is comprised primarily of a gas or gases, such as nitrogen, that are inert to oxidation. Thus, the oxidant stream may comprise dry air containing about 21 mole percent molecular oxygen and substantial amounts of nitrogen.

The presence or absence of carbon dioxide in the oxidant stream is not seen to be especially critical, and may thus vary within a broad range, from substantially no carbon dioxide, to that amount of carbon dioxide normally found in fresh air (about 0.05 wt. %), or up to about 1 wt. %, or up to 2 wt. %, or up to 4 wt. %, or even greater amounts.

In the processes according to the invention, the oxidation reaction proceeds at elevated temperatures and pressures, so that at least a portion of the reaction mixture is in the liquid phase. During oxidation, the time-averaged and volume-averaged temperature of the reaction medium may be maintained, for example, in the range from about 125° C. to about 210° C., or from about 130° C. to about 190° C., or from 135° C. to 170° C. The overhead pressure above the reaction medium may, for example, be maintained in the range of from about 1 to about 40 bar gauge (barg), or from about 2 to about 20 barg, or from 2 to 8 barg.

We have found according to the invention that relatively moderate oxidation temperatures may also be used, such as from about 130° C. to about 180° C., for example, to help reduce the extent of carbon oxides formation, believed to represent in part the extent of solvent burn. The processes of the invention thus are particularly well suited for oxidizing p-xylene at relatively moderate temperatures, as already described.

The catalyst compositions employed in the processes of the invention comprise cobalt atoms, manganese atoms, bismuth atoms, and bromine atoms, supplied by any suitable means, as further described below. In a preferred embodiment, the catalyst compositions consist essentially of cobalt atoms, manganese atoms, bismuth atoms, and bromine atoms. The catalyst composition is typically soluble in the solvent under reaction conditions, or it is soluble in the reactants fed to the oxidation zone. Preferably, the catalyst composition is soluble in the solvent at 40° C. and 1 atm, and is soluble in the solvent under the reaction conditions.

The cobalt atoms may be present, for example, in an amount of at least 500 ppm, or at least 800 ppm, or at least 1,200 ppm, or from about 500 ppm to about 6,000 ppm, or from 800 ppm to 5,000 ppm, or from 1,200 ppm to 4,000 ppm, in each instance with respect to the weight of the liquid in the reaction medium. The cobalt atoms may be provided in ionic form as inorganic cobalt salts, such as cobalt bromide, cobalt nitrate, or cobalt chloride, or organic cobalt compounds such as cobalt salts of aliphatic or aromatic acids having 2-22 carbon atoms, including cobalt acetate, cobalt octanoate, cobalt benzoate, cobalt acetylacetonate, and cobalt naphthalate.

The weight amounts of each of cobalt, manganese, bismuth, and bromine are based on the atomic weight of the atoms, whether or not the atoms are in elemental form or in ionic form. For example, the amount of cobalt refers to the amount of cobalt atoms, whether elemental or ionic, and not the amount of cobalt acetate. The stated concentrations of catalyst components are based on the quantity of catalyst components in the liquid portion of the reaction medium in the oxidation reactor. The catalyst component concentrations may be measured, for example, by sampling the oxidation reactor.

The oxidation state of cobalt when added as a compound to the reaction mixture is not limited, and includes both the +2 and +3 oxidation states.

The manganese atoms may be provided as one or more inorganic manganese salts, such as manganese borates, manganese halides, manganese nitrates, or organometallic manganese compounds such as the manganese salts of lower aliphatic carboxylic acids, including manganese acetate, and manganese salts of beta-diketonates, including manganese acetylacetonate. Manganese of the catalyst composition may be present in a concentration from about 20 to about 425 ppm, or from 20 to 300 ppm, or from 20 to 200 ppm.

The bismuth may be provided as bismuth oxide or as one or more inorganic bismuth salts, such as bismuth bromide, bismuth chloride or bismuth oxychloride, or as bismuth salts of lower aliphatic carboxylic acids, including bismuth acetate, bismuth 2-ethylhexanoate, or organometallic bismuth compounds such as triphenylbismuth. The bismuth atoms of the catalyst composition may be present in an amount of at least 25 ppm, or at least 50 ppm, or at least 100 ppm, up to about 1,100 ppm, or up to about 1,500 ppm, or up to about 1,800 ppm. Suitable ranges may thus vary from about 25 ppm to about 1,800 ppm, or from 50 ppm to 1,500 ppm, or from 100 ppm to 1,100 ppm.

The bromine component may be added as elemental bromine, in combined form, or as an anion. Suitable sources of bromine include hydrobromic acid, sodium bromide, ammonium bromide, potassium bromide, tetrabromoethane, benzyl bromide, alpha-bromo-p-toluic acid, and bromoacetic acid. Hydrobromic acid, sodium bromide, or alpha-bromo-p-toluic acid may be preferred bromine sources. Bromine may thus be present in an amount ranging from about 750 to about 6,000 ppm, based on the total liquid in the reaction medium, or from 1,000 ppm to 5,500 ppm, or from 1,200 to 5,000 ppm, each with respect to the total weight of liquid in the reaction medium.

According to the invention, the relative amounts of elements in the catalyst composition are selected so as to achieve acceptable conversion, while limiting acid burn. Thus, the weight ratio of cobalt atoms to bromine atoms may be, for example, from about 0.6 to about 10, or from 0.7 to 8, or from 0.8 to 5.

According to the invention, the ratio of cobalt atoms to manganese atoms may vary within a wide ranges, such as from about 5 to about 400, or from about 10 to about 300, or from about 15 to about 150. In various other embodiments, the ratio of cobalt atoms to manganese atoms will be at least about 10, or at least 12, or at least 15, or at least 18, up to about 150, or up to about 200, or up to about 300, or up to about 400.

Other organic or non-metallic catalyst components can be included in the catalyst composition of the invention, or the processes may be carried out in the substantial absence of additional organic or non-metallic catalysts. For example, the catalyst composition may include a source of pyridine. The pyridine component of the catalyst composition may be added to a primary oxidation reactor or to post oxidation reactors. The pyridine component can be in the form of pyridine per se or in the form of a compound of pyridine.

Further, the processes according to the invention may be carried out in the presence of, or in the substantial absence of, one or more aldehydes or ketones.

Further, the processes according to the invention may be carried out in the presence of additional metal atoms, or in the substantial absence thereof, so long as the catalyst composition comprises cobalt atoms, manganese atoms, and bismuth atoms, with bromine atoms provided as a promoter. Such additional metals may include, but not be limited to, molybdenum, sodium, potassium, copper, hafnium, chromium, cerium, iron, tungsten, zirconium, vanadium, and palladium. When present, sodium may be used, for example, in an amount up to about 500 ppm, or up to about 1,000 ppm, or up to about 1,500 ppm, or from about 10 ppm to about 1,500 ppm, or from about 100 ppm to about 1,000 ppm, for example, based on the total weight of liquid in the reaction medium.

The catalyst composition can be formed by adding each source to the oxidation reactor separately, in sequence, or simultaneously, or a prepared composition may be added to the oxidation reactor, and in either case, the addition may be made as an initial batch or continuously during the course of the oxidation reaction. The catalyst composition prepared as a batch may be dissolved in the solvent to form a catalyst feed followed by adding the catalyst feed to the primary oxidation reactor. Each component, or the catalyst composition batch, can be added to the primary oxidation reactor before, during, or after addition of the solvent. In a continuous process, the catalyst components or the catalyst composition may be added simultaneous with the solvent feed, or in the solvent feed, or separately metered as required for fresh make-up.

After the initial charge of catalyst composition in a continuous process, the residual mother liquor from the primary oxidation may supply a portion of the necessary catalyst components to the primary oxidation reactor by partial displacement of the primary oxidation mother liquor with fresh solvent. The remainder can be made up with a continuous fresh feed of make-up catalyst.

In the processes according to the invention, the extent of solvent burned and rendered unusable as a recycle stream may be reduced relative to typical processes. While the absolute amount of solvent burn in the present invention may be reduced, this reduction is not achieved at the expense of acceptable conversion. Obtaining a low amount of carbon oxides formation might be achieved by running the reaction at low oxidation temperatures or using a less active catalyst, but this typically results in lowered conversion and increased quantities of intermediates. The processes of the invention have the advantage of maintaining a low carbon oxides formation while minimizing the impact on conversion.

Thus, in a preferred embodiment, the amount of carbon oxides formation (in total moles of CO and CO₂, expressed as COx per mole of para- and/or meta-substituted dialkyl aromatic compounds fed to the reactor) is no more than about 1.2 moles COx/mole, or no more than about 0.6, or no more than about 0.3 mole COₓ per mole of para- and/or meta-substituted dialkyl aromatic compounds fed to the reactor.

Thus, in a process in accordance with the present invention, p-xylene, in an amount, for example, from about 2 to about 15 wt. %, based on the weight of liquid in the reaction medium, is combined with acetic acid, and an oxygen-containing gas, at a temperature from about 130° C. to about 180° C., using a catalyst composition comprising cobalt atoms, manganese atoms, and bismuth atoms, with bromine atoms provided as a promoter, wherein the bismuth atoms are present in an amount of at least 25 ppm, or at least 50 ppm, up to about 1,200 ppm, or up to about 1,500 ppm.

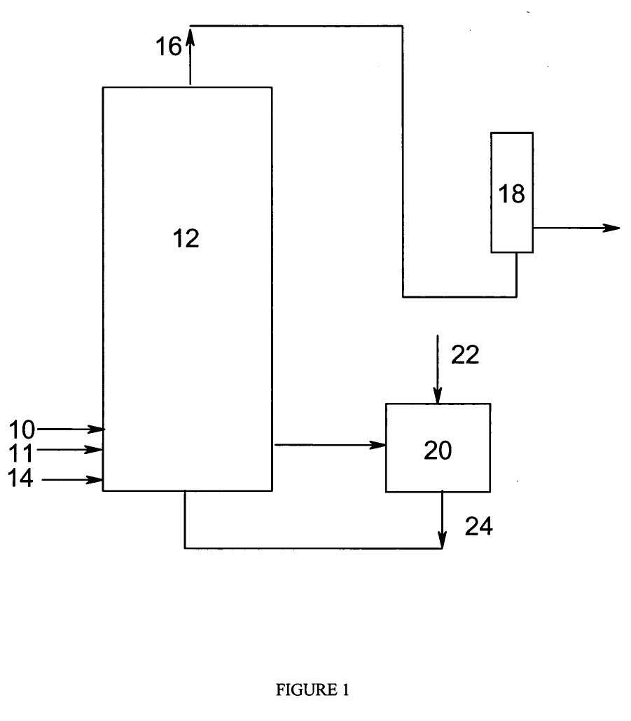

An embodiment of the invention will now be described referring to the accompanying FIG. 1, in which p-xylene is introduced via conduit 10 into primary oxidation reactor 12, and aqueous acetic acid solvent having dissolved therein the catalyst composition of the invention fed through line 11 to the reactor 12. If desired, the p-xylene, solvent, and catalyst composition charges may be fed to reactor 12 at a plurality of points, or fed together through one line. An oxygen-containing gas under pressure is introduced near the bottom of the reactor 12 via conduit 14. The preferred oxygen-containing gas is air or oxygen-enriched air. The flow rate of the oxygen-containing gas to reactor 12 is controlled to maintain between about 2 and 9 volume percent oxygen (calculated on a dry, solvent free basis) in the off-gas which exits the reactor via conduit 16. The reactants in reactor 12 are maintained at an elevated pressure of about 50 to 175 psia to maintain a contained, volatizable reaction medium substantially in the liquid state at the reaction temperature of about 130° C. to about 180° C.

During the course of the oxidation reaction, exothermic heat of reaction and water generated by the oxidation of p-xylene are removed from reactor 12 by vaporization of a portion of the liquid reaction medium. These vapors, known as reactor off-gas, comprise vaporized acetic acid solvent, about 5 to 30 weight percent water, and oxygen-depleted process gas containing minor amounts of decomposition products including catalyst residue, as well as additional carbon dioxide and carbon monoxide generated by the decomposition of acetic acid. The reactor off-gas passes upwardly through the reactor 12 and is conveyed via conduit 16 to the lower portion of water removal column 18 for distillation and recovery of the acetic acid back to the primary oxidation reactor. The crude reaction mixture is discharged from the primary oxidation reactor to a solid/liquid separator 20 into which is fed fresh acetic acid through line 22 to exchange the mother liquor discharged through line 24. The mother liquor containing acetic acid and the catalyst composition is subjected to conventional purification and purging techniques to recover and recycle the catalyst composition to the primary oxidation reactor 12.

Suitable dialkyl aromatic compounds useful as reactor feed-mixture components or ingredients in the methods of the present invention include para- or meta-substituted dialkyl benzenes and para- or meta-substituted naphthalenes such as m-xylene, p-xylene, 2,6-dimethylnaphthalene, 2,7-dimethylnaphthalene and 2,6-diisopropylnaphthalene. The respective aromatic carboxylic acid products of these alkyl aromatic compounds are isophthalic acid, terephthalic acid (TPA), and 2,6- and 2,7-naphthalenedicarboxylic acids. The processes of the invention can be used to produce TPA and isophthalic acid, and are particularly well suited for the production of benzenedicarboxylic and naphthalenedicarboxylic acids, especially TPA.

Suitable aqueous aliphatic acid solvents useful in the methods of the invention are those that are readily volatilizable at the reaction temperatures. Among such solvents are aqueous solutions of C₂ to C₆ monocarboxylic acids, e.g., acetic acid, propionic acid, n-butyric acid, isobutyric acid, n-valeric acid, trimethylacetic acid, caprioic acid, and mixtures thereof. Preferably, the volatilizable monocarboxylic aliphatic acid solvent is an aqueous acetic acid solution.

Further description of the oxidation of alkyl aromatics to benzenepolycarboxylic acids may be found in the “Phthalic Acids and Other Benzenepolycarboxylic Acids” entry of Kirk-Othmer Encyclopedia of Chemical Technology, Vol 18, 4th ed., (1995) pp. 991-1043, the relevant portions of which are incorporated herein by reference.

The measure of 4-carboxybenzaldehyde (4-CBA) in the product mixture from the oxidation of p-xylene to terephthalic acid, being an incomplete oxidation product, is understood to indicate the degree of conversion achieved, with lower 4-CBA levels indicating higher conversion. Of course, when the reactant comprises m-xylene, the corresponding carboxybenzaldehyde will be 3-carboxybenzaldehyde. When not otherwise specified, the one or more carboxybenzaldehydes will be those which correspond to the para- or meta-substituted dialkyl aromatic reactant provided to the reaction medium.

As described, the acetic acid solvent is decomposed, to some extent, in a side reaction to produce mainly carbon dioxide and carbon monoxide. The total oxidative decomposition products were estimated in the examples by measuring the number of moles of carbon dioxide and carbon monoxide exiting in the oxidizer vent gas. To achieve satisfactory results for the oxidation process, the amount of carbon oxides formation should be minimal while the rate of conversion to dicarboxylic acid is maximized (the concentration of carboxybenzaldehydes (CBA) in the product mixture is low). Thus, the amount of carboxybenzaldehydes found in the oxidizer filtrate is a measure of the rate of the oxidation, and the amount of carbon oxides in the vent gas is a measure of the cost of the oxidation process, in terms of acid burn and oxidative degradation of aromatics.

The invention has been described in detail with particular reference to preferred embodiments thereof, but it will be understood that variations and modifications can be effected within the spirit and scope of the invention. Moreover, all patents, patent applications (published or unpublished, foreign or domestic), literature references or other publications noted above are incorporated herein by reference for any disclosure pertinent to the practice of this invention.

### EXAMPLES

### Examples 1-47

In Examples 1-47, a solution containing the catalyst components in concentrations described in Table 1 was prepared and 60 grams of the solution was transferred to the autoclave. Cobalt, manganese and bismuth were provided as cobalt(II) acetate hydrate, manganese(II) acetate hydrate and bismuth acetate, respectively. Bromine was added as hydrobromic acid and sodium bromide in concentrations found in Table 1. The autoclave was pressurized with approx. 20 bar nitrogen (ca. 290 psi) and then the catalyst solution was heated up to the reaction temperature under stirring at 2000 rpm.

At reaction temperature, the air flow diluted down to 10% oxygen in nitrogen for safety reasons was started at a total gas flow of 1.13 NL/min and the reaction pressure was adjusted to the desired total pressure to ensure a fairly constant oxygen partial pressure. Then the para-xylene feeding (140 μL/min) was started (this is t=0 for the reaction time). Thirty seconds after the start of the para-xylene feeding, 0.25 mmol peracetic acid in 1.5 mil acetic acid was introduced into the solution to start the reaction.

The para-xylene feeding was stopped 30 min after the start. Subsequently the gas flow was stopped, and the system was cooled down. The autoclave was then depressurized and the reaction solution was filtered to isolate the CTA. The yield of the filtrate was recorded. The CTA was washed two times with 25 ml of acetic acid (96%) and then once with 40 ml methanol. The washed CTA was dried at 60° C. and then weighed.

The off-gas was analyzed with respect to CO₂ and CO by ND-IR (ABB, Advanced Optima) and O₂ by a paramagnetism detection system (Hartmann & Braun, Magnos 6G). The composition of the solid isolated and the filtrate was determined by high-pressure liquid chromatography.

TABLE 1
Results from semi-batch reaction Examples 1-47 performed as described above.

| | | | | | | | Br | | | |
|---|---|---|---|---|---|---|---|---|---|---|
| | | | | | | | (as | Br (as | | 4- |
| | T | P | H2O | Co | Mn | Bi | HBr) | NaBr) | COx | CBA(s) |
| Example | ° C. | barg | % | ppm | ppm | ppm | ppm | ppm | mol/mol | ppm |
| 1 | 135 | 17.4 | 4 | 2000 | 90 | 1390 | 1800 | 370 | 0.04 | 8820 |
| 2 | 135 | 17.4 | 4 | 4000 | 180 | 1390 | 1800 | 2530 | 0.05 | 9010 |
| 3 | 145 | 18.0 | 8 | 3000 | 130 | 1050 | 1800 | 1450 | 0.08 | 3480 |
| 4 | 135 | 17.5 | 8 | 4000 | 180 | 700 | 1800 | 2530 | 0.05 | 5450 |
| 5 | 135 | 17.5 | 4 | 2000 | 90 | 1390 | 1800 | 370 | 0.05 | 5550 |
| 6 | 145 | 18.0 | 4 | 4000 | 180 | 1050 | 1800 | 2530 | 0.09 | 37380 |
| 7 | 145 | 18.0 | 6 | 4000 | 180 | 1050 | 1800 | 2530 | 0.09 | 3570 |
| 8 | 155 | 18.7 | 8 | 2000 | 90 | 700 | 1800 | 370 | 0.12 | 790 |
| 9 | 145 | 17.9 | 6 | 3000 | 130 | 1050 | 1800 | 1450 | 0.00 | 24040 |
| 10 | 145 | 17.9 | 6 | 3000 | 130 | 1050 | 1800 | 1450 | 0.00 | 18750 |
| 11 | 145 | 18.0 | 6 | 3000 | 130 | 1050 | 1800 | 1450 | 0.08 | 23560 |
| 12 | 155 | 18.7 | 8 | 4000 | 180 | 1390 | 1800 | 2530 | 0.18 | 5820 |
| 13 | 145 | 18.0 | 6 | 3000 | 130 | 1390 | 1800 | 1450 | 0.08 | 17610 |
| 14 | 135 | 17.5 | 8 | 2000 | 90 | 700 | 1800 | 370 | 0.05 | 69040 |
| 15 | 155 | 18.7 | 8 | 4000 | 180 | 700 | 1800 | 2530 | 0.14 | 8670 |
| 16 | 135 | 17.5 | 6 | 3000 | 130 | 1050 | 1800 | 1450 | 0.05 | 48960 |
| 17 | 155 | 18.7 | 8 | 2000 | 90 | 1390 | 1800 | 370 | 0.11 | 11380 |
| 18 | 135 | 17.5 | 8 | 4000 | 180 | 1390 | 1800 | 2530 | 0.05 | 59860 |
| 19 | 145 | 18.0 | 6 | 3000 | 130 | 700 | 1800 | 1450 | 0.09 | 16950 |
| 20 | 155 | 18.7 | 6 | 3000 | 130 | 1050 | 1800 | 1450 | 0.14 | 7560 |
| 21 | 135 | 17.5 | 8 | 2000 | 90 | 1390 | 1800 | 370 | 0.05 | 64060 |
| 22 | 145 | 18.0 | 6 | 2000 | 90 | 1050 | 1800 | 370 | 0.07 | 29190 |
| 23 | 155 | 18.6 | 4 | 2000 | 90 | 1390 | 1800 | 370 | 0.17 | 4350 |
| 24 | 160 | 19.0 | 4 | 1800 | 80 | 30 | 1950 | 0 | 0.19 | 3560 |
| 25 | 160 | 19.0 | 4 | 1800 | 80 | 700 | 1950 | 0 | 0.17 | 3190 |
| 26 | 140 | 17.7 | 4 | 1800 | 80 | 1050 | 1800 | 150 | 0.06 | 34220 |
| 27 | 155 | 18.6 | 4 | 2700 | 120 | 610 | 1800 | 1130 | 0.16 | 6150 |
| 28 | 140 | 17.7 | 4 | 3600 | 160 | 1050 | 1800 | 2100 | 0.08 | 23600 |
| 29 | 170 | 19.9 | 4 | 1800 | 80 | 1050 | 1800 | 150 | 0.34 | 1400 |
| 30 | 155 | 18.6 | 4 | 4000 | 180 | 1390 | 1800 | 2534 | 0.19 | 5940 |
| 31 | 155 | 18.6 | 4 | 2000 | 90 | 1390 | 1800 | 370 | 0.16 | 11400 |
| 32 | 145 | 18.0 | 4 | 3000 | 130 | 1390 | 1800 | 1450 | 0.09 | 49970 |
| 33 | 145 | 18.0 | 4 | 3000 | 130 | 1050 | 1800 | 1450 | 0.09 | 5780 |
| 34 | 135 | 17.4 | 4 | 3000 | 130 | 1050 | 1800 | 1450 | 0.06 | 26860 |
| 35 | 145 | 18.0 | 4 | 3000 | 130 | 1050 | 1800 | 1450 | 0.08 | 14800 |
| 36 | 155 | 18.6 | 4 | 2000 | 90 | 700 | 1800 | 370 | 0.12 | 53400 |
| 37 | 145 | 18.0 | 4 | 3000 | 130 | 1050 | 1800 | 1450 | 0.12 | 4580 |
| 38 | 135 | 17.4 | 4 | 2000 | 90 | 700 | 1800 | 370 | 0.05 | 17560 |
| 39 | 155 | 18.6 | 4 | 3000 | 130 | 1050 | 1800 | 1450 | 0.19 | 42610 |
| 40 | 155 | 18.6 | 4 | 4000 | 180 | 700 | 1800 | 2530 | 0.20 | 18360 |
| 41 | 135 | 17.4 | 4 | 4000 | 180 | 700 | 1800 | 2530 | 0.06 | 16190 |
| 42 | 145 | 18.0 | 4 | 2000 | 90 | 1050 | 1800 | 370 | 0.07 | 44230 |
| 43 | 145 | 18.0 | 4 | 3000 | 130 | 700 | 1800 | 1450 | 0.09 | 17720 |
| Comp. Ex. 44 | 160 | 19.0 | 4 | 1800 | 80 | 0 | 1950 | 0 | 0.17 | 4410 |
| Comp. Ex. 45 | 155 | 18.6 | 3 | 1600 | 400 | 0 | 1800 | 0 | 0.08 | 26150 |
| Comp. Ex. 46 | 155 | 18.8 | 11 | 3000 | 400 | 0 | 1800 | 0 | 0.09 | 20910 |
| Comp. Ex. 47 | 155 | 18.8 | 11 | 1600 | 30 | 0 | 1800 | 0 | 0.11 | 17360 |

COₓ is the sum of CO and CO₂ measured in the off-gas and listed as mol of COₓ per mol of p-xylene (pX) fed.
4CBA(S) is the concentration of 4CBA found in the dry isolated solid;
Results for Comparative Example 44 are averages of 12 experiments.

The many features and advantages of the invention are apparent from the detailed specification and, thus, it is intended by the appended claims to cover all such features and advantages of the invention which fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

We claim:

1. A process for the liquid phase oxidation of one or more of a para- or a meta-substituted dialkyl aromatic compound to produce a corresponding aromatic dicarboxylic acid product, the process comprising combining in a reaction medium the one or more para- or meta-substituted dialkyl aromatic compound, a solvent mixture comprising water and a saturated organic acid having from 2-4 carbon atoms, and an oxygen-containing gas, at a temperature from about 130° C. to about 180° C., in the presence of a catalyst composition comprising cobalt, manganese, bismuth, and bromine.

2. The process according to claim 1, wherein the one or more para- or meta-substituted dialkyl aromatic compound comprises one or more of p-xylene or m-xylene.

3. The process according to claim 1, wherein the one or more para- or meta-substituted dialkyl aromatic compound comprises p-xylene in an amount of at least 90 wt. % with respect to a total amount of dialkyl aromatic compounds provided to the reaction medium.

4. The process according to claim 1, wherein the one or more para- or meta-substituted dialkyl aromatic compound comprises p-xylene in an amount of at least 99 wt. % with respect to a total amount of dialkyl aromatic compounds provided to the reaction medium.

5. The process according to claim 1, wherein the one or more para- or meta-substituted dialkyl aromatic compound comprises p-xylene and the corresponding aromatic dicarboxylic acid product comprises terephthalic acid.

6. The process according to claim 1, wherein the bismuth is present in an amount from about 50 ppm to about 1,500 ppm, with respect to the weight of the liquid in the reaction medium.

7. The process according to claim 1, wherein the weight ratio of cobalt to manganese in the reaction medium is from about 10 to about 300.

8. The process according to claim 1, wherein the weight ratio of cobalt to bromine in the reaction medium is from about 0.6 to about 10.

9. The process according to claim 1, wherein the saturated organic acid comprises acetic acid.

10. The process according to claim 1, wherein the bismuth is provided as one or more of: bismuth oxide, bismuth bromide, bismuth chloride, bismuth oxychloride, bismuth acetate, bismuth 2-ethylhexanoate, or triphenylbismuth.

11. The process according to claim 1, wherein the bismuth is provided as bismuth acetate.

12. The process according to claim 1, wherein the oxygen-containing gas comprises air.

13. The process according to claim 1, wherein the cobalt is provided in an amount from about 500 ppm to about 6,000 ppm, with respect to the weight of liquid in the reaction medium.

14. The process according to claim 1, wherein the manganese is provided in an amount from about 20 ppm to about 425 ppm, with respect to the weight of liquid in the reaction medium.

15. The process according to claim 1, wherein the bromine is provided in an amount from about 750 ppm to about 6,000 ppm, with respect to the weight of liquid in the reaction medium.

16. The process according to claim 1, wherein the process produces no more than about 1.2 moles COₓ per mole of the total para- and meta-substituted dialkyl aromatic compounds provided to the reaction medium.

17. The process according to claim 1, wherein the process produces no more than about 3 wt. % of the corresponding carboxybenzaldehyde based on the total weight of the corresponding aromatic dicarboxylic acid product produced.

18. A process for the liquid phase oxidation of one or more of a para- or a meta-substituted dialkyl aromatic compound to produce a corresponding aromatic dicarboxylic acid product, the process comprising combining in a reaction medium the one or more para- or meta-substituted dialkyl aromatic compound, a solvent mixture comprising water and a saturated organic acid having from 2-4 carbon atoms, and an oxygen-containing gas, at a temperature from about 130° C. to about 180° C., in the presence of a catalyst composition comprising cobalt, manganese, bismuth, and bromine, wherein the process results in less of the corresponding carboxybenzaldehyde in the reaction medium than would be present in the absence of bismuth.
